(19) United States
(12) Patent Application Publication
(10) Pub. No.: US 2024/0423174 A1
(43) Pub. Date: Dec. 26, 2024

(54) ANIMAL MODEL OF CD19 CHIMERIC ANTIGEN RECEPTOR (CAR)-T CELL THERAPY FOR LEUKEMIA COMPLICATED BY CYTOKINE RELEASE SYNDROME (CRS), AND PREPARATION METHOD AND USE THEREOF

(71) Applicant: UNION HOSPITAL TONGJI MEDICAL COLLEGE HUST, Wuhan City (CN)

(72) Inventors: Heng Mei, Wuhan City (CN); Yu Hu, Wuhan City (CN); Zhaozhao Chen, Wuhan City (CN); Yingying Li, Wuhan City (CN)

(21) Appl. No.: 18/439,283

(22) Filed: Feb. 12, 2024

(30) Foreign Application Priority Data
Jun. 25, 2023 (CN) 2023107512940

(51) Int. Cl. A01K 67/027; C12N 5/0783

(52) U.S. Cl. CPC A01K 67/027; C12N 5/0636; A01K 2227/105; A01K 2267/0331; C12N 2500/32; C12N 2501/2302

(57) ABSTRACT

Provide is an animal model of a chimeric antigen receptor (CAR)-T cell therapy for leukemia complicated by cytokine release syndrome (CRS), and a preparation method and use thereof. The preparation method of an animal model of a CD19 CAR-T cell therapy for leukemia complicated by CRS includes: preparing a CD19 CAR-T cell line: conducting cell separation of peripheral blood mononuclear cells (PBMCs), magnetic beads cell sorting of T cells, activation of the T cells, lentiviral infection, and amplification of CD19 CAR-T cells; and constructing CRS animal models after high-dose and low-dose CD19 CAR-T infusions: implanting a Nalm-6 cell line into a living animal to allow leukemia tumor burden to obtain a leukemia tumor-burdened living animal, and injecting the CD19 CAR-T cell line into the leukemia tumor-burdened living animal. The present disclosure further provides new use of the animal model of a CD19 CAR-T cell therapy for leukemia complicated by CRS.

### CROSS REFERENCE TO RELATED APPLICATION

This patent application claims the benefit and priority of Chinese Patent Application No. 2023107512940, filed with the China National Intellectual Property Administration on Jun. 25, 2023, the disclosure of which is incorporated by reference herein in its entirety as part of the present application.

### TECHNICAL FIELD

The present disclosure relates to the field of leukemia research, and in particular relates to an animal model of a CD19 chimeric antigen receptor (CAR)-T cell therapy for leukemia complicated by cytokine release syndrome (CRS), and a preparation method and use thereof.

### BACKGROUND

CD19 chimeric antigen receptor (CAR)-T cell therapy shows a desirable therapeutic effect on leukemia, especially lymphocytic leukemia. CD19 CAR-T cells have become an effective treatment for relapsed/refractory hematological malignancies, but are also accompanied by the occurrence of cytokine release syndrome (CRS) while achieving significant efficacy. The CRS is the most common side effect of CD19 CAR-T therapy. About 50% to 100% of patients develop CRS, and up to 48% of which develop CRS greater than or equal to Grade 3 that is characterized by significantly elevated IL-6, vascular leakage, hypotension, pulmonary edema, cardiac insufficiency, liver and kidney failure, neurological toxicity, disseminated intravascular coagulation, and other multiple organ failures and even death. These seriously affect the efficacy and prognosis of CD19 CAR-T therapy. Currently, clinical intervention methods for CRS are limited, mainly including cytokine antagonists and glucocorticoids, which cannot avoid the occurrence of serious adverse reactions. Therefore, it is urgent to find the key factors in the occurrence of CRS by constructing a CRS model and to block the occurrence of CRS in a timely manner, thus improving the safety and feasibility of CD19 CAR-T cell therapy. At present, only patent CN114032208A has constructed an in vitro cytokine storm model. However, due to a large number of cells and cytokines involved in the CRS, it is difficult for in vitro cell models to truly simulate the occurrence of CRS, and the demands of models in the medical field cannot be achieved for studying inflammation induced by specific cytokines.

### SUMMARY

In view of the above problems, the present disclosure provides an animal model of a CD19 CAR-T cell therapy for leukemia complicated by CRS, and a preparation method and use thereof. The present disclosure is mainly intended to solve the current difficulty in obtaining a CD19 CAR-T model for treating leukemia complicated by CRS in vitro.

To solve the mentioned technical problems, the present disclosure adopts the following technical solution.

A first aspect of the present disclosure relates to a preparation method of an animal model of a CD19 CAR-T cell therapy for leukemia complicated by CRS, including the following steps:
preparing a CD19 CAR-T cell line: conducting cell separation of peripheral blood mononuclear cells (PBMCs), magnetic beads cell sorting of T cells, activation of the T cells, lentiviral infection, and amplification of CD19 CAR-T cells; and
constructing CRS animal models after high-dose and low-dose CD19 CAR-T infusions:
implanting a Nalm-6 cell line into a living animal to allow leukemia tumor burden to obtain a leukemia tumor-burdened living animal, and injecting the CD19 CAR-T cell line into the leukemia tumor-burdened living animal.

Regarding each cell line, a number of CD19 CAR-T cells in the CD19 CAR-T cell line is 50 to 200 times that of Nalm-6 cells in the Nalm-6 cell line. Further, the Nalm-6 cell line has 10⁵ to 10⁶ of Nalm-6 cells; and the CD19 CAR-T cell line has 10⁷ to 10⁸ of CD19 CAR-T cells. More specifically, the Nalm-6 cell line has 10⁵, 2×10⁵, 5×10⁵, or 10⁶ of the Nalm-6 cells; and the number of the CD19 CAR-T cells in the CD19 CAR-T cell line is 100 times that of Nalm-6 cells in the Nalm-6 cell line.

The living animal is a SCID/Beige mouse, which has SCID/Beige severe combined immunodeficiency, lacks functional T cells and B cells and shows NK cell functional defects, but exhibits sound macrophage and dendritic cell functions.

A construction process of the Nalm-6 cell line includes the following steps: taking a Nalm6 cell line growing in a logarithmic phase as a target cell line, placing cells of the target cell line into a culture dish containing a culture solution Polybrene, adding a GFP-luc double-labeled lentivirus solution into the culture dish and shaking uniformly, transferring the culture dish into an incubator to allow incubation, and sorting GFP-positive Nalm6 cells by flow cytometry to allow propagation culture to obtain a GFP-luc double-labeled Nalm6 cell line.
conducting the cell separation of the PBMCs: collecting peripheral blood of a subject to be treated and separating mononuclear cells;
conducting the activation of the T cells: washing a culture flask once with an X-VIVO 15 medium pre-added with IL-2, sodium pyruvate, and glutamine, discarding the X-VIVO 15 medium by aspiration and adding a culture medium, adding an inactivated autologous plasma, transferring T cells obtained from the magnetic beads cell sorting of the T cells into the culture flask, mixing evenly, and incubating in a carbon dioxide incubator;
conducting the lentiviral infection: adding a lentivirus solution into the culture flask to be infected, mixing gently with a recombinant medium, continuing the incubating in the carbon dioxide incubator, and centrifuging to obtain T cells after the lentivirus infection; and
conducting the amplification of the CD19 CAR-T cells: transferring the T cells after the lentivirus infection into a new culture flask to allow culture until a number of cells is greater than 10⁸.

A second aspect of the present disclosure relates to an animal model of a CD19 CAR-T cell therapy for leukemia complicated by CRS prepared by the preparation method.

A third aspect of the present disclosure relates to new use of the animal model of a CD19 CAR-T cell therapy for leukemia complicated by CRS.

First, the use of the animal model of a CD19 CAR-T cell therapy for leukemia complicated by CRS is provided in screening a drug for treating leukemia. The animal model can be directly used to screen drugs that are effective in controlling the leukemia complicated by CRS with CD19 CAR-T therapy, thereby providing a basis for clinical screening of effective drugs. Of course, the use generally appears in some commercial studies, and its main purpose is also to study the therapeutic effects of different drugs on CD19 CAR-T therapy of leukemia complicated by CRS.

Second, the use of the animal model of a CD19 CAR-T cell therapy for leukemia complicated by CRS is provided in preparation of a drug for controlling leukemia. The animal model is mainly used to screen ingredients that have a therapeutic effect on the CD19 CAR-T therapy of leukemia complicated by CRS, and these ingredients can be used as active ingredients of the drugs, thereby making the corresponding drugs have an effect of achieving the CD19 CAR-T therapy of leukemia complicated by CRS.

The present disclosure has following beneficial effects:

The present disclosure constructs a simple and reliable CRS mouse model. In the present disclosure, an in vivo model capable of simulating clinical CRS to the greatest extent is constructed by using mice with severe combined immunodeficiency, inoculating high- and low-load tumor cells, and infusing CD19 CAR-T cells of different doses. The model also has excellent accuracy and consistency performance, and can provide a scientific research guidance for clinical exploration of more effective intervention measures.

### BRIEF DESCRIPTION OF THE DRAWINGS

FIG. 1 shows in vivo imaging of mice with high and low tumor burdens before and after CD19 CAR-T cell reinfusion;

FIG. 2 shows body weight changes of the mice after CD19 CAR-T cell reinfusion;

FIG. 3 shows changes of an IL-6 level in the mice after CD19 CAR-T cell reinfusion;

FIG. 4 shows changes of an IL-1B level in the mice after CD19 CAR-T cell reinfusion;

FIG. 5 shows fibrinogen levels in three groups of the mice after CD19 CAR-T cell reinfusion;

FIG. 6 shows HE staining of liver, lung, and brain tissues in three groups of the mice after CD19 CAR-T reinfusion;

FIG. 7 shows infiltration of CD19 CAR-T cells in liver, lungs, brain, and spleen of the mice in each group after reinfusion; and

FIG. 8 shows tumor burden states of three groups: 1*10⁵, 2*10⁵, and 5*10⁵.

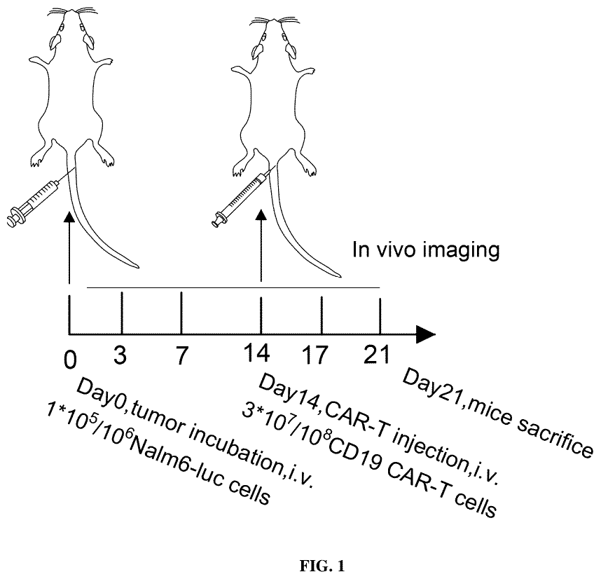

### DETAILED DESCRIPTION OF THE EMBODIMENTS

The present disclosure will be further described below with reference to the accompanying drawings.

### Step 1: Preparation of a CD19 CAR-T Cell Line

### A. Cell Separation of PBMCs

1) A 100 mL bag of fresh blood was collected from a patient, irradiated with UV light and sterilized with alcohol, put in a center of a biological safety cabinet, divided into two 50 mL centrifuge tubes A and B, and a 1 mL of a blood sample was spread on a plate to allow bacterial examination.

2) The centrifuge tubes A and B were centrifuged at 400 g for 8 min, an upper plasma was transferred into two 50 mL centrifuge tubes C and D, inactivated in a 56° C. water bath for 30 min, cooled to a room temperature, and transferred into a biological safety cabinet. 20 mL of a physiological saline were added to each of the centrifuge tubes A and B, and gently blown evenly.

3) 15 mL of a Ficoll lymphocyte separation solution was added to each of the two 50 mL centrifuge tubes E and F, tube bodies were tilted, cell suspensions in the centrifuge tubes A and B were slowly added along a tube wall into the centrifuge tubes E and F, the centrifuge tubes were gradually stood up upright during the liquid addition, such that a resulting mixed liquid was always kept on a surface of the Ficoll solution and a liquid interface was clear and stable.

4) The centrifuge tubes E and F were centrifuged at 400 g for 18 min (speed up 1, speed down 0), taken out carefully and smoothly, a junctional cell layer in each centrifuge tube was collected with a Pasteur dropper and distributed evenly into 2 50 mL centrifuge tubes G and H.

5) The centrifuge tubes G and H were filled with physiological saline to 50 mL, the tube caps were tightened, and the tubes were turned upside down several times, and centrifuged at 130 g for 5 min at room temperature. After centrifugation, the contents were observed; if there were few remaining red blood cells, next step was directly conducted. If there were many remaining red blood cells, the red blood cells were lysed once and centrifuged at 130 g for 5 min at room temperature.

6) The centrifuge tubes G and H were transferred to the center of the biological safety cabinet, a supernatant was discarded and a resulting pellet was resuspended in X-VIVO 15 medium, and centrifuged at 130 g for 5 min at room temperature.

### B. Magnetic Beads Cell Sorting of T Cells

1) After centrifugation in the previous step, a resulting supernatant was discarded and a resulting pellet was resuspended in X-VIVO 15 medium; a small amount of an obtained cell suspension was collected to allow counting with Trypan blue, while the remaining cell suspension was centrifuged at 300 g for 10 min to remove a supernatant. According to the cell counting results, a resulting pellet was resuspended in Buffer to make a cell concentration to 2.5×10⁸ cells/mL.

2) 40 μL of a cell suspension (containing 1×10⁷ PBMC) was placed in a centrifuge tube, added with 10 μL of Pan T Cell Biotin Antibody Cocktail, mixed well and incubated in a 4° C. refrigerator for 5 min.

3) 30 μL of buffer and 20 μL of PanT Cell MicroBead Cocktail were added to the cells in sequence, mixed well, and incubated in a 4° C. refrigerator for 10 min.

4) An MS-type separation column was fixed on a magnetic stand and rinsed once with 0.5 mL of Buffer; after replacing the centrifuge tube with a new one below, a magnetic bead-labeled cell suspension was slowly added to the separation column for sorting; a liquid passed through the separation column was collected, centrifuged at 130 g for 5 min at room temperature, and a resulting supernatant was discarded to obtain a precipitate, namely the T cells.

### C. Activation of T Cells

1) A T25 cell culture flask pre-coated (at least 24 h in advance) with CD3 and CD28 was taken, all the coating solution was discarded, washed once with 10 mL of X-VIVO 15 medium pre-added with IL-2, sodium pyruvate, and glutamine, added with 10 mL of a same medium after removing the old one, and added with 1 mL of an inactivated autologous plasma.

2) A small amount of medium was aspirated to resuspend the T cell pellet, and the pellet was transferred to a culture flask to make a final cell concentration of 1×10⁶ cells/mL. The cells were gently mixed and placed in a carbon dioxide incubator to start incubation, which was recorded as day 0 (DO).

### D. Lentiviral Infection

1) On D2, 12×10⁷ lentivirus was thawed at room temperature. According to the counting results of T cell culture, 4×10⁷ of the T cells were added to a new culture flask for infection. The remaining cells were continued to be cultured and used as a control group for CD19 CAR-T cell function testing.

2) The lentivirus solution returned to room temperature and then added into the culture flask to be infected. A recombinant medium was added to adjust a cell density to 1×10⁶ cells/mL. The cells were gently mixed and placed in a carbon dioxide incubator to continue incubation.

3) The cells were observed and counted every day, fluids were replenished in time to maintain a cell density at 2×10⁶ cells/mL.

4) On D5, a resulting cell culture was transferred into a centrifuge tube, centrifuged at 130 g for 5 min at room temperature, a resulting supernatant was discarded, and the remaining precipitate was washed twice with an appropriate amount of physiological saline.

### E. Amplification of CD19 CAR-T Cells

1) The washed T cells were transferred into a new culture flask, a cell density was adjusted to 5×10⁵ to 2×10⁶ cells/mL, the cells were mixed well, 1 mL of a cell solution was spread on a plate for bacterial examination, and the rest cells were placed into a carbon dioxide incubator for incubation.

2) The cells were observed and counted every day, and the flask was rotated as appropriate. When the number of cells was greater than 1×10⁸, an appropriate amount of cells was collected for killing experiments, factor release experiments, PCR detection, and flow cytometry.

### Step 2: Construction of Mouse Models with High and Low Leukemia Tumor Burdens

15 specific pathogen-free grade SCID/Beige male mice, 8 weeks old, weighing 20 g to 22 g, were purchased from Beijing Vital River Laboratory Animal Technology Co., Ltd. The mice were randomly divided into a healthy control group (n=3), a low tumor burden group+low CD19 CAR-T dose group (low+low, n=4), a high tumor burden group+low CD19 CAR-T dose group (high+low, n=4), and a high tumor burden group+high CD19 CAR-T dose group (high+high, n=4). After one week of adaptive feeding in an isolation package laboratory, the Nalm-6 cell lines at 1*10⁵ (low tumor burden group) and 1*10⁶ (high tumor burden group) were injected into the tail vein; in vivo imaging of the mice was taken on D7, D14, and D18 after tumor inoculation to evaluate tumor formation.

### Step 3: Construction of CRS Mouse Models after High- and Low-Dose CD19 CAR-T Infusions

On day 18 after tumor inoculation, a difference between the high and low tumor burdens was clearly observed through small animal in vivo imaging; in the low CD19 CAR-T dose group, 1*10⁷ human CD19 CAR-T cells were injected into the tail vein; while in the high CD19 CAR-T dose group, 1*10⁸ human CD19 CAR-T cells were injected into the tail vein. At 12 h, 24 h, 48 h, 72 h, 96 h, 120 h, 144 h, and 168 h after the infusion of CD19 CAR-T cells, blood was collected from the retroorbital vein, the body weight of mice was measured, and a body weight change trend chart was drawn; each of the blood samples was centrifuged at 4° C., 3,000 rpm for 15 min, and an upper plasma was collected for detection of IL-6 and IL-1B. 168 h after CD19 CAR-T cell reinfusion, the mice were anesthetized with 1% sodium pentobarbital; the eyeballs were removed to collect blood, and the liver, kidney, lung, brain, spleen and other organs were collected for HE pathological staining to observe the inflammatory infiltration; a human CD8 antibody was used for immunohistochemical staining to observe the infiltration of CD19 CAR-T cells.

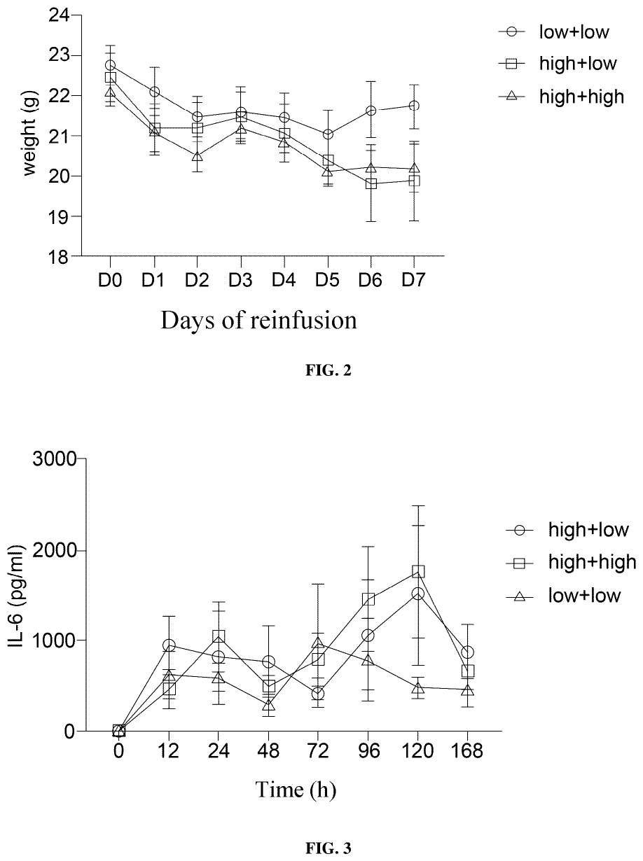

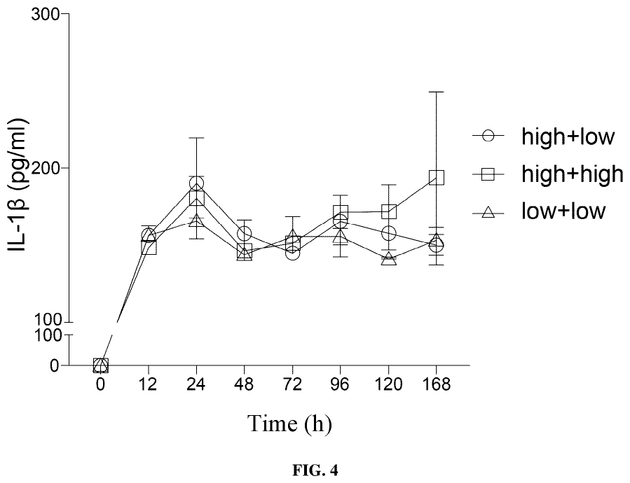

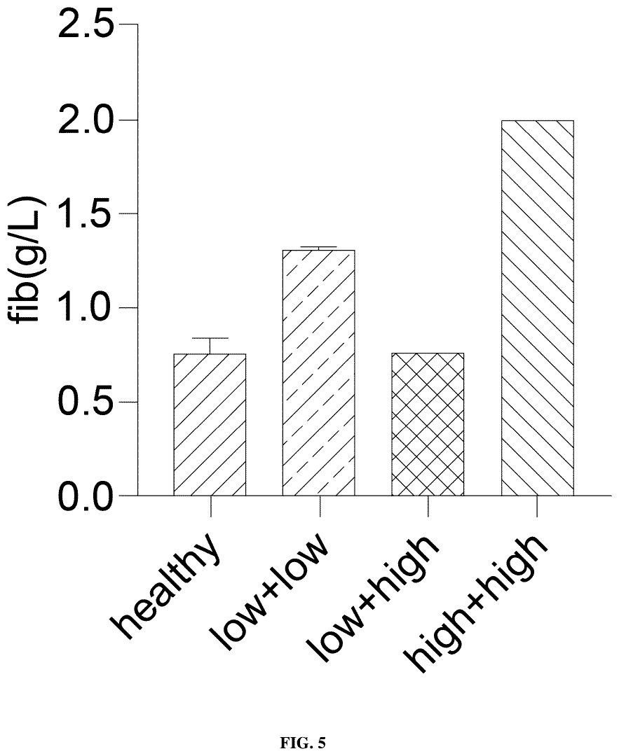

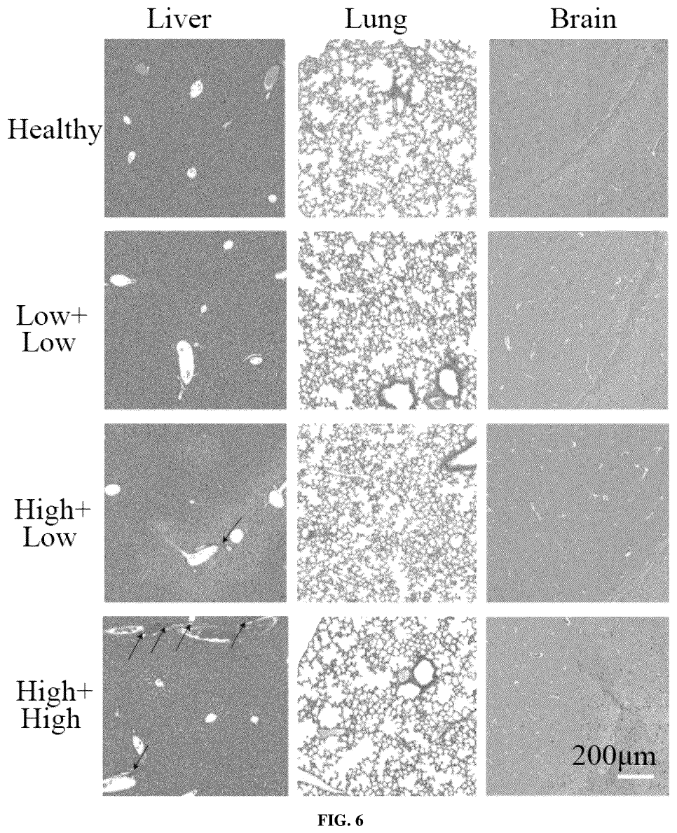

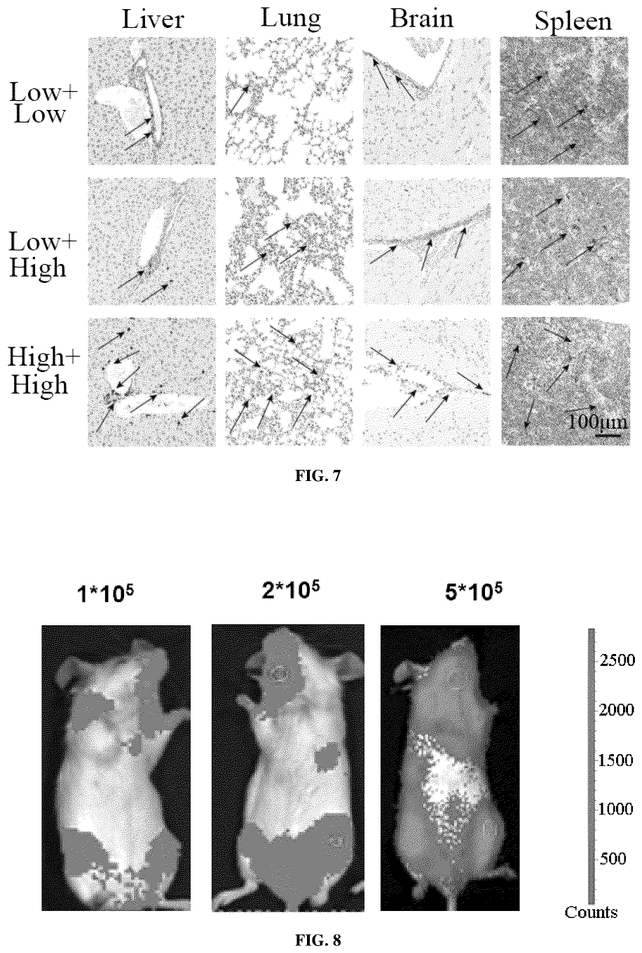

FIG. 2 showed that when CRS was produced in vivo, a state of high inflammation was presented, which might lead to high metabolic performance and thus weight loss; when the level of CRS decreased, the weight increased again. FIG. 3 and FIG. 4 showed that IL-6 and IL-1B were typical cytokines used in CD19 CAR-T therapy of leukemia complicated by CRS, and both played a pro-inflammatory role. FIG. 5 demonstrated that elevated fibrinogen levels represented a high inflammatory state in the body. FIG. 6 showed that in the HE staining, arrows in the liver section represented the infiltration of CD19 CAR-T cells, indicating the activation and amplification of CD19 CAR-T cells in vivo. FIG. 7 further showed the distribution and infiltration of CD19 CAR-T cells in various organs detected by immunohistochemistry, where arrows indicated the activation and amplification of CD19 CAR-T cells in vivo.

CRS occurs because specifically targeted CD19 CAR-T cells are activated and massively expanded by tumor cells in vivo after reinfusion, releasing a large amount of cytokines and chemokines, such as IL-6, IFN-γ, and GM-CSF. These cytokines further activate bystander immune cells such as monocytes and macrophages, and further release cytokines such as IL-1B, IL-6, and IL-10. However, a specific pathogenesis and occurrence process of the CRS remain unclear due to limited clinical treatments for CRS and difficulties in establishing in vitro models. In the present disclosure, the SCID-Beige mouse used is a severe combined immunodeficiency mouse, which has SCID/Beige severe combined immunodeficiency, lacks functional T cells and B cells and shows NK cell functional defects, but exhibits sound macrophage and dendritic cell functions has. The mouse also shows high tumor formation rate and is suitable for xenograft transplantation. CD19 CAR-T cell amplification and tumor burden are most closely related to the occurrence of CRS. Therefore, in the present disclosure, high and low leukemia tumor burden models are constructed and high and low doses of CD19 CAR-T cells are reinfused; the CD19 CAR-T cells are activated in large quantities after contact with tumor cells, and then amplified to release varying degrees of cytokines. These cytokines turn activate macrophages in mice, causing the CRS. On the 21st day after NSG mice were injected with a corresponding amount of Nalm6-luc cell line into the tail vein, the tumor burden was detected using a small animal in vivo imaging system. The results showed that the tumor burdens in the three groups of 1*10⁵, 2*10⁵, and 5*10⁵ increased with an increase in the number of Nalm6-luc cells injected, indicating that the high and low burden tumor models were positively correlated with the number of cells injected into the tail vein during modeling. Accordingly, continuing to increase the number of cells infused into the tail vein during modeling might help to increase the difference between the high and low tumor burden groups.

It will be clear to those skilled in the art that various modifications to the above examples can be made without departing from the general spirit and concept of the present disclosure. These modifications shall all fall within the protection scope of the present disclosure. The claimed protection schemes of the present disclosure shall be determined by the claims.

What is claimed is:

1. A preparation method of an animal model of a CD19 chimeric antigen receptor (CAR)-T cell therapy for leukemia complicated by cytokine release syndrome (CRS), comprising the following steps:
preparing a CD19 CAR-T cell line: conducting cell separation of peripheral blood mononuclear cells (PBMCs), magnetic beads cell sorting of T cells, activation of the T cells, lentiviral infection, and amplification of CD19 CAR-T cells; and
constructing CRS animal models after high-dose and low-dose CD19 CAR-T infusions:
implanting a Nalm-6 cell line into a living animal to allow leukemia tumor burden to obtain a leukemia tumor-burdened living animal, and injecting the CD19 CAR-T cell line into the leukemia tumor-burdened living animal.

2. The preparation method of an animal model of a CD19 CAR-T cell therapy for leukemia complicated by CRS according to claim 1, wherein a number of CD19 CAR-T cells in the CD19 CAR-T cell line is 50 to 200 times that of Nalm-6 cells in the Nalm-6 cell line.

3. The preparation method of an animal model of a CD19 CAR-T cell therapy for leukemia complicated by CRS according to claim 2, wherein the Nalm-6 cell line has 10⁵ to 10⁶ of the Nalm-6 cells; and the CD19 CAR-T cell line has 10⁷ to 10⁸ of the CD19 CAR-T cells.

4. The preparation method of an animal model of a CD19 CAR-T cell therapy for leukemia complicated by CRS according to claim 3, wherein the Nalm-6 cell line has 10⁵, 2×10⁵, 5×10⁵, or 10⁶ of the Nalm-6 cells.

5. The preparation method of an animal model of a CD19 CAR-T cell therapy for leukemia complicated by CRS according to claim 4, wherein the number of the CD19 CAR-T cells in the CD19 CAR-T cell line is 100 times that of the Nalm-6 cells in the Nalm-6 cell line.

6. The preparation method of an animal model of a CD19 CAR-T cell therapy for leukemia complicated by CRS according to claim 1, wherein the living animal is a SCID/Beige mouse; and
a construction process of the Nalm-6 cell line comprises: taking a Nalm6 cell line growing in a logarithmic phase as a target cell line, placing cells of the target cell line into a culture dish containing a culture solution Polybrene, adding a GFP-luc double-labeled lentivirus solution into the culture dish and shaking uniformly, transferring the culture dish into an incubator to allow incubation, and sorting GFP-positive Nalm6 cells by flow cytometry to allow propagation culture to obtain a GFP-luc double-labeled Nalm6 cell line.

7. The preparation method of an animal model of a CD19 CAR-T cell therapy for leukemia complicated by CRS according to claim 1, specifically comprising:
conducting the cell separation of the PBMCs: collecting peripheral blood of a subject to be treated and separating mononuclear cells;
conducting the activation of the T cells: washing a culture flask once with an X-VIVO 15 medium pre-added with IL-2, sodium pyruvate, and glutamine, discarding the X-VIVO 15 medium by aspiration and adding a culture medium, adding an inactivated autologous plasma, transferring T cells obtained from the magnetic beads cell sorting of the T cells into the culture flask, mixing evenly, and incubating in a carbon dioxide incubator;
conducting the lentiviral infection: adding a lentivirus solution into the culture flask to be infected, mixing gently with a recombinant medium, continuing the incubating in the carbon dioxide incubator, and centrifuging to obtain T cells after the lentivirus infection; and
conducting the amplification of the CD19 CAR-T cells: transferring the T cells after the lentivirus infection into a new culture flask to allow culture until a number of cells is greater than 10⁸.

8. An animal model of a CD19 CAR-T cell therapy for leukemia complicated by CRS prepared by the preparation method according to claim 1.

9. An animal model of a CD19 CAR-T cell therapy for leukemia complicated by CRS prepared by the preparation method according to claim 2.

10. An animal model of a CD19 CAR-T cell therapy for leukemia complicated by CRS prepared by the preparation method according to claim 3.

11. An animal model of a CD19 CAR-T cell therapy for leukemia complicated by CRS prepared by the preparation method according to claim 4.

12. An animal model of a CD19 CAR-T cell therapy for leukemia complicated by CRS prepared by the preparation method according to claim 5.

13. An animal model of a CD19 CAR-T cell therapy for leukemia complicated by CRS prepared by the preparation method according to claim 6.

14. An animal model of a CD19 CAR-T cell therapy for leukemia complicated by CRS prepared by the preparation method according to claim 7.
